(19) United States
(12) Patent Application Publication
(10) Pub. No.: US 2009/0239880 A1
(43) Pub. Date: Sep. 24, 2009

(54) COMBINATIONS OF MONOAMINE REUPTAKE INHIBITORS AND POTASSIUM CHANNEL ACTIVATORS

(76) Inventors: Ulrik Svane Sorensen, Soborg (DK); Birgitte L. Eriksen, Farum (DK); Lene Teuber, Vaerlose (DK); Dan Peters, Malmo (SE); Dorte Strobaek, Farum (DK); Tina Holm Johansen, Smorum (DK); Palle Christophersen, Ballerup (DK); John Paul Redrobe, Rodovre (DK)

Correspondence Address: BIRCH STEWART KOLASCH & BIRCH, PO BOX 747, FALLS CHURCH, VA, 22040-0747, US

(21) Appl. No.: 12/306,890

(22) PCT Filed: Jul. 2, 2007
(86) PCT No.: PCT/EP07/56624

§ 371 Date: Dec. 29, 2008

Related U.S. Application Data
(60) Provisional application No. 60/818,326, filed on Jul. 5, 2006.

(30) Foreign Application Priority Data
Jul. 3, 2006 (DK) PA 2006 00910

(51) Int. Cl. A61K 31/497; C07D 401/04; A61K 31/454

(52) U.S. Cl. 514/254.06; 546/199; 544/370; 514/322

(57) ABSTRACT

This invention provides pharmaceutical compositions comprising therapeutically effective amounts of a monoamine reuptake inhibitor and an SK inhibitor. In another aspect the invention provides novel benzoimidazole derivatives for use according to the invention.

### TECHNICAL FIELD

This invention provides pharmaceutical compositions comprising therapeutically effective amounts of a monoamine reuptake inhibitor and an SK inhibitor. In another aspect the invention provides novel benzoimidazole derivatives for use according to the invention.

### BACKGROUND ART

Mono-aminergic (MA) neurons are located in limited number in distinct brain areas: Dopaminergic neurons in the ventral tegmental area (VTA) and substantia nigra compacta (SNc), serotonergic neurons in the raphe nucleus and the noradrenergic neurons in the locus coeruleus. All MA neurons exert wide-ranging modulatory neurotransmission in the brain, with the dopaminergic systems projecting to nucleus accumbens, prefrontal cortex and the limbic system (VTA) and the striatum (SNc). The raphe serotonergic neurons and the locus coeruleus noradrenaline neurons project both to the whole forebrain.

The monoaminergic neurotransmission is central in the treatment of a large number of psychiatric and neurological disorders, such as depression, bipolar disorder, attention deficit hyperactivity disorder (ADHD), schizophrenia, Parkinsons disease, Huntingtons disease, etc. The molecular targets involved are post- and pre-synaptic MA receptors as well as the presynaptic MA uptake systems, which are pivotal in the control of the intensity and the timing of MA signaling.

Depression is treated with a plethora of drugs acting on the presynaptic MA uptake systems: the oldest of these compounds, the tricyclic antidepressants like imipramine, are also the least selective, inhibiting all MA uptake systems as well as some MA receptors, and having a number of adverse effects in the clinic. Second generation compounds, i.e. selective serotonin reuptake inhibitors (SSRIs) like Fluoxetine and Paroxetine, are widely used and have substantial less classical side effects than the tricyclic compounds (reduced sexual drive remains a problem), although the prolonged time to action in combination with a significant proportion of non-responders limits their therapeutic use. Third generation MA inhibitors represents compounds with various selectivity profiles from selective noradrenaline uptake inhibitors (SNRIs), as Reboxetine, to dual acting (SA and NA) inhibitors as Venlafaxine and Duloxetine. Triple action compounds (SA, NA, DA) for depression have not yet been marketed, although such compounds are generally supposed to have a faster onset of action.

Strengthening of MA transmission by re-uptake inhibitors is an established antidepressant principle in the clinic. Preclinically, depression models include the acute despair models (the tail suspension and the forced swim tests) as well as more chronic models (the chronic mild stress model and the olfactory bulbectomy model). Furthermore, supporting pharmacological models exists, showing interaction with the various MA systems (serotonin syndrome by nialamide facilitation of locomotor activity, noradrenaline syndrome by the reboxetine prevention of tetrabenazine induced ptosis, and dopamine syndromes as methylphenidate induced stereotypy and locomotor activity).

Increased MA transmission can be attained by increasing the electrical firing or the firing pattern of MA neurons. In general, MA neurons fire irregularly, determined by the relative excitatory and inhibitory presynaptic drives, as well as their endogenous rhythmic activity. Action potentials arriving at the presynaptic terminal increases MA release much more effectively than action potentials coming in single firing pattern: Differential afferent modulation of VTA firing pattern strongly regulates the balance between tonic and phasic dopamine transmission in the nucleous accumbens.

Blocking small-conductance calcium-activated potassium channels (SK channels) with the selective bee poison peptide constituent, apamin, also effectively switches dopaminergic neurons from regular pacemaker-like firing to a highly bursting mode, both in vitro and in vivo after local administration. However, due to poor blood brain barrier permeability of apamin, this compound is not suitable for behavioural testing.

### SUMMARY OF THE INVENTION

The present invention provides a new principle for the treatment of a large number of psychiatric and neurological diseases based on altered MA signalling in various brain regions. The invention focuses on the combined therapeutic effect of an activity at all or a subset of MA uptake mechanisms and at the same blocking one or more of the presynaptic SK channels (SK1, SK2, and preferably SK3, which is the predominant SK subtype expressed in MA neurons). This therapeutic effect may be accomplished using a monoamine reuptake inhibitor simultaneously with an SK inhibitor, i.e. by using two separate compounds. It may, however, also be accomplished using one therapeutically active ingredient having this dual therapeutic activity.

Also according to the present invention, we have found small molecule organic compounds with potent (nM) dual MA inhibiting and SK channel inhibiting actions.

Therefore, in its first aspect, the invention provides pharmaceutical compositions comprising a therapeutically effective amount of an active pharmaceutical ingredient (API) selected from A) a monoamine reuptake inhibitor; and B) an SK inhibitor; together with one or more adjuvants, excipients, carriers and/or diluents.

In another aspect the invention provides benzoimidazole derivatives of Formula I

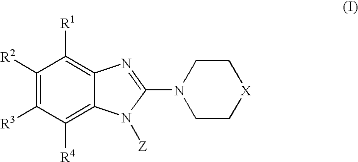

an isomer thereof or a mixture of its isomers, or a pharmaceutically acceptable salt thereof, wherein

R¹, R²2, R³ and R⁴, independently of each other, represent hydrogen, halo, trifluoromethyl, trifluoromethoxy, cyano, alkyl, alkoxy, amino, N-alkyl-amino or N,N-dialkyl-amino;

Z represents hydrogen, alkyl or benzyl, which benzyl may optionally be substituted one or more times with halo, trifluoromethyl, trifluoromethoxy, cyano, alkyl, alkoxy, amino, N-alkyl-amino and/or N,N-dialkyl-amino;

X represents CH-A′, N-A′, or C=A″; wherein

A′ represents a group of Formula Ia or Ib:

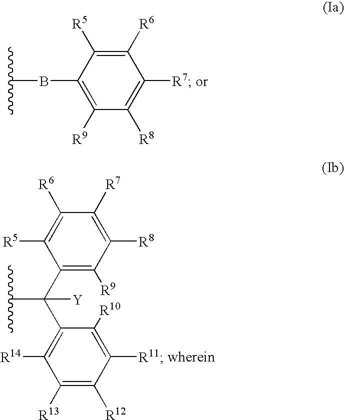

B represents CH₂, Q or S;

Y represents hydrogen, fluoro, hydroxy or alkoxy; and

R⁵, R⁶, R⁷, R⁸, R⁹, R¹⁰, R¹¹, R¹², R¹³ and R¹⁴, independently of each other, represent hydrogen, halo, trifluoromethyl, trifluoromethoxy, cyano, alkyl, alkoxy, amino, N-alkyl-amino or N,N-dialkyl-amino; and

A″ represents a group of Formula Ic:

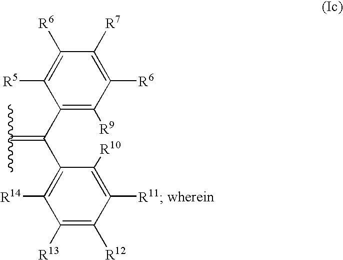

R⁵, R⁶, R⁷, R⁸, R⁹, R¹⁰, R¹¹, R¹², R¹³ and R¹⁴, independently of each other, represent hydrogen, halo, trifluoromethyl, trifluoromethoxy, cyano, alkyl, alkoxy, amino, N-alkyl-amino or N,N-dialkyl-amino.

Other objects of the invention will be apparent to the person skilled in the art from the following detailed description and examples.

### DETAILED DISCLOSURE OF THE INVENTION

### Pharmaceutical Compositions

In its first aspect the invention provides pharmaceutical compositions comprising a therapeutically effective amount of an active pharmaceutical ingredient (API) selected from A) a monoamine reuptake inhibitor; and

B) an SK inhibitor;

together with one or more adjuvants, excipients, carriers and/or diluents.

The carrier(s) must be “acceptable” in the sense of being compatible with the other ingredients of the formulation and not harmful to the recipient thereof.

In a preferred embodiment the active pharmaceutical ingredients (API) show biological activity at the sub-micromolar level (i.e. below 1 μM), preferably at the low nanomolar level (i.e. below 0.1 μM).

In another preferred embodiment the monoamine reuptake inhibitor is a dopamine uptake inhibitor, in particular bupropion, sertraline, nomifensine, or mazindol, or vanoxerine, or a noradrenaline uptake inhibitor, in particular Amoxapine, Atomoxetine, reboxetine, or a serotonin reuptake inhibitor, in particular Citalopram, Escitalopram, Fluoxetine, fluvoxamine maleate, Paroxetine, Sertraline or Zimelidine.

In a more preferred embodiment the monoamine reuptake inhibitor is a selective serotonin reuptake inhibitor (SSRI) selected from the group consisting of citalopram (Celexa, Cipramil, Emocal, Sepram), escitalopram oxalate (Lexapro, Cipratex, Esertia), fluoxetine (Prozac, Fontex, Seromex, Seronil, Sarafem, Fluctin (EUR)), fluvoxamine maleate (Luvox, Faverin), paroxetine (Paxil, Seroxat, Aropax, Deroxat) and sertraline (Zoloft, Lustral, Serlain).

In a third preferred embodiment the SK inhibitor for use according to the invention is a benzoimidazole derivative of Formula I as defined below.

In yet another preferred embodiment the pharmaceutical composition of the invention comprises a compound having the dual activity of a monoamine reuptake inhibitor and an SK inhibitor as the only active pharmaceutical ingredient (API).

In a more preferred embodiment the API having the dual activity of a monoamine reuptake inhibitor and an SK inhibitor is a benzoimidazole derivative of Formula I as defined below.

In another more preferred embodiment the API having the dual activity of a monoamine reuptake inhibitor and an SK inhibitor show a dual biological activity at the sub-micromolar level (i.e. below 1 μM), preferably at the low nanomolar level (i.e. below 0.1 μM).

While a chemical compound of the invention for use in therapy may be administered in the form of the raw chemical compound, it is preferred to introduce the active ingredient, optionally in the form of a physiologically acceptable salt, or in the form of a prodrug, in a pharmaceutical composition together with one or more adjuvants, excipients, carriers, buffers, diluents, and/or other customary pharmaceutical auxiliaries.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be prepared by any person skilled in the art, by use of standard methods and conventional techniques, appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect.

### Benzoimidazole Derivatives

In another aspect the invention provides novel benzoimidazole derivatives. The benzoimidazole derivatives of the invention may be characterised by Formula I

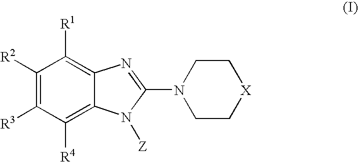

an isomer thereof or a mixture of its isomers, or a pharmaceutically acceptable salt thereof, wherein

R¹, R², R³ and R⁴, independently of each other, represent hydrogen, halo, trifluoromethyl, trifluoromethoxy, cyano, alkyl, alkoxy, amino, N-alkyl-amino or N,N-dialkyl-amino; and

Z represents hydrogen, alkyl or benzyl, which benzyl may optionally be substituted one or more times with halo, trifluoromethyl, trifluoromethoxy, cyano, alkyl, alkoxy, amino, N-alkyl-amino and/or N,N-dialkyl-amino;

X represents CH-A′, N-A′, or C=A″; wherein

A′ represents a group of Formula Ia or Ib:

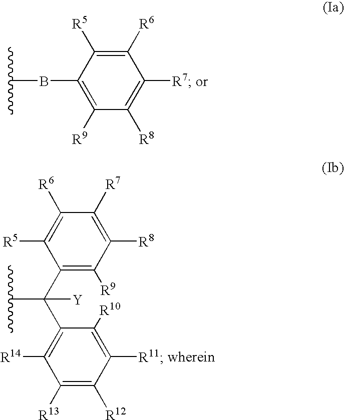

B represents CH₂, O or S;

Y represents hydrogen, fluoro, hydroxy or alkoxy; and

R⁵, R⁶, R⁷, R⁸, R⁹, R¹⁰, R¹¹, R¹², R¹³, and R¹⁴, independently of each other, represent hydrogen, halo, trifluoromethyl, trifluoromethoxy, cyano, alkyl, alkoxy, amino, N-alkyl-amino or N,N-dialkyl-amino; and

A″ represents a group of Formula Ic:

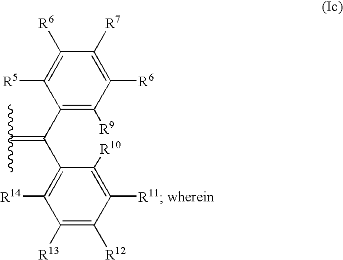

R⁵, R⁶, R⁷, R⁸, R⁹, R¹⁰, R¹¹, R¹², R¹³ and R¹⁴, independently of each other, represent hydrogen, halo, trifluoromethyl, trifluoromethoxy, cyano, alkyl, alkoxy, amino, N-alkyl-amino or N,N-dialkyl-amino.

In a particular embodiment, however, the benzoimidazole derivative of the invention is not
2-(4-Benzylpiperidin-1-yl)-5,6-dimethoxy-1H-benzoimidazole;
2-[4-(4-Chlorobenzyl)piperazin-1-yl]-1-propyl-1H-benzoimidazole;
2-[4-(4-Chlorobenzyl)piperazin-1-yl]-1-isopropyl-1H-benzoimidazole;
2-[4-(2,5-Dimethyl-benzyl)piperazin-1-yl]-6-trifluoromethyl-1H-benzoimidazole;
6-Trifluoromethyl-2-[4-(2-trifluoromethyl-benzyl)-piperazin-1-yl]-1H-benzoimidazole;
2-[4-(4-tert-Butylbenzyl)piperazin-1-yl]-6-trifluoromethyl-1H-benzoimidazole;
2-[4-(2,6-Dichlorobenzyl)piperazin-1-yl]-6-trifluoromethyl-1H-benzoimidazole;
2-(4-Benzhydrylpiperazin-1-yl)-1-(4-chlorobenzyl)-1H-benzoimidazole;
2-(4-Benzylpiperazin-1-yl)-1-pentyl-1H-benzoimidazole;
2-(4-Benzhydrylpiperazin-1-yl)-1-benzyl-1H-benzoimidazole;
2-(4-Benzhydrylpiperazin-1-yl)-1-methyl-1H-benzoimidazole; or
2-(4-Benzylpiperazin-1-yl)-1H-benzoimidazole.

In a first preferred embodiment the benzoimidazole derivative of the invention is a compound of Formulas I-IV, wherein R¹, R², R³ and R⁴, independently of each other, represent hydrogen, halo, trifluoromethyl, trifluoromethoxy, cyano, alkyl, alkoxy, amino, N-alkyl-amino or N,N-dialkyl-amino.

In a more preferred embodiment two of R¹, R², R³ and R⁴, independently of each other, represent halo, trifluoromethyl, trifluoromethoxy, cyano, alkyl, alkoxy, amino, N-alkyl-amino or N,N-dialkyl-amino; and the remaining two of R¹, R², R³ and R⁴ all represent hydrogen. In an even more preferred embodiment the two of R¹, R², R³ and R⁴ representing halo, trifluoromethyl, trifluoromethoxy, cyano, alkyl, alkoxy, amino, N-alkyl-amino or N,N-dialkyl-amino are R¹ and R², or R¹ and R³, or R² and R³.

In another more preferred embodiment two of R¹, R², R³ and R⁴, independently of each other, represent halo, trifluoromethyl, trifluoromethoxy or cyano; and the remaining two of R¹, R², R³ and R⁴ all represent hydrogen. In an even more preferred embodiment the two of R¹, R², R³ and R⁴ representing halo, trifluoromethyl, trifluoromethoxy or cyano are R¹ and R², or R¹ and R³, or R² and R³.

In still another more preferred embodiment one of R¹, R², R³ and R⁴, independently of each other, represent halo, trifluoromethyl, trifluoromethoxy, cyano, alkyl, alkoxy, amino, N-alkyl-amino or N,N-dialkyl-amino; and the remaining three of R¹, R², R³ and R⁴ all represent hydrogen. In an even more preferred embodiment the one of R¹, R², R³ and R⁴ representing halo, trifluoromethyl, trifluoromethoxy, cyano, alkyl, alkoxy, amino, N-alkyl-amino or N,N-dialkyl-amino is R¹ or R² or R³.

In yet another more preferred embodiment one of R¹, R², R³ and R⁴, independently of each other, represent halo, trifluoromethyl, trifluoromethoxy or cyano; and the remaining three of R¹, R², R³ and R⁴ all represent hydrogen. In an even more preferred embodiment the one of R¹, R², R³ and R⁴ representing halo, trifluoromethyl, trifluoromethoxy or cyano R¹ or R² or R³.

In a most preferred embodiment R¹, R², R³ and R⁴ all represent hydrogen.

In a second preferred embodiment the benzoimidazole derivative of the invention is a compound of Formulas I-IV, wherein Z represents hydrogen, alkyl or benzyl, which benzyl may optionally be substituted one or more times with halo, trifluoromethyl, trifluoromethoxy, cyano, alkyl, alkoxy, amino, N-alkyl-amino and/or N,N-dialkyl-amino.

In a more preferred embodiment Z represents hydrogen, alkyl or benzyl, which benzyl may optionally be substituted one or two times with halo, trifluoromethyl and/or trifluoromethoxy.

In an even more preferred embodiment Z represents hydrogen, alkyl or benzyl, which benzyl may optionally be substituted one or two times with halo and/or trifluoromethyl.

In a still more preferred embodiment Z represents hydrogen, alkyl or benzyl, which benzyl may optionally be substituted one or two times with fluoro, chloro and/or trifluoromethyl.

In a yet more preferred embodiment Z represents hydrogen or alkyl.

In a further more preferred embodiment Z represents benzyl, optionally be substituted one or two times with fluoro, chloro and/or trifluoromethyl.

In a third preferred embodiment the benzoimidazole derivative of the invention is a compound of Formulas I-IV, wherein X represents CH-A′ or N-A′, and A′ is as defined above.

In a more preferred embodiment X represents CH-A′, and A′ is as defined above.

In another more preferred embodiment X represents N-A′, and A′ is as defined above.

In a fourth preferred embodiment the benzoimidazole derivative of the invention is a compound of Formula II,

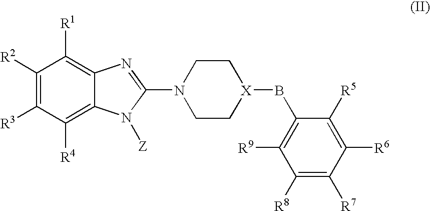

an isomer thereof or a mixture of its isomers, or a pharmaceutically acceptable salt thereof, wherein

R¹, R², R³, R⁴, R⁵, R⁶, R⁷, R⁸ and R⁹, independently of each other, represent hydrogen, halo, trifluoromethyl, trifluoromethoxy, cyano, alkyl, alkoxy, amino, N-alkyl-amino or N,N-dialkyl-amino;

Z represents hydrogen, alkyl or benzyl, which benzyl may optionally be substituted one or more times with halo, trifluoromethyl, trifluoromethoxy, cyano, alkyl, alkoxy, amino, N-alkyl-amino and/or N,N-dialkyl-amino;

X represents CH or N; and

B represents CH₂, O or S.

In a more preferred embodiment R¹, R², R³, R⁴, R⁵, R⁶, R⁷, R⁸ and R⁹, independently of each other, represent hydrogen, halo, trifluoromethyl, trifluoromethoxy, cyano, alkyl, alkoxy, amino, N-alkyl-amino or N,N-dialkyl-amino.

In an even more preferred embodiment R¹, R², R³, R⁴, R⁵, R⁶, R⁷, R⁸ and R⁹, independently of each other, represent hydrogen, halo or trifluoromethyl.

In a still more preferred embodiment R¹, R², R³, R⁴, R⁵, R⁶, R⁷, R⁸ and R⁹, independently of each other, represent hydrogen or halo, and in particular fluoro or chloro.

In another more preferred embodiment Z represents hydrogen, alkyl or benzyl, which benzyl may optionally be substituted one or more times with halo, trifluoromethyl, trifluoromethoxy, cyano, alkyl, alkoxy, amino, N-alkyl-amino and/or N,N-dialkyl-amino.

In an even more preferred embodiment Z represents hydrogen or benzyl, which benzyl may optionally be substituted one or two times with halo, in particular fluoro or chloro, and/or trifluoromethyl.

In a third more preferred embodiment X represents CH or N. In an even more preferred embodiment X represents CH. In another even more preferred embodiment X represents N.

In a fourth more preferred embodiment B represents CH₂, O or S. In an even more preferred embodiment B represents O or S. In another even more preferred embodiment B represents CH₂. In a third even more preferred embodiment

B represents 0. In a fourth even more preferred embodiment B represents S.

In a most preferred embodiment the benzoimidazole derivative of the invention is
2-(4-Benzylpiperidin-1-yl)-1H-benzoimidazole;
2-(4-Benzylpiperidin-1-yl)-1-[4-chloro-3-(trifluoromethyl)benzyl]-1H-benzoimidazole;
2-(4-Benzylpiperidin-1-yl)-1-(3,4-difluorobenzyl)-1H-benzoimidazole
2-[4-(3,4-Dichlorophenoxy)piperidin-1-yl]-1H-benzoimidazole;
2-[4-(3,4-Dichlorophenylsulfanyl)piperidin-1-yl]-1H-benzoimidazole;
2-[4-(3,4-Dichlorobenzyl)piperidin-1-yl]-1H-benzoimidazole;
2-(4-Benzylpiperazin-1-yl)-1H-benzoimidazole;
1-(3,4-Difluorobenzyl)-2-[4-(3,4-difluorobenzyl)piperazin-1-yl]-1H-benzoimidazole; or
2-[4-(3,4-Difluorobenzyl)piperidin-1-yl]-1H-benzoimidazole;

or an enantiomers or a mixture of its enantiomers, or a pharmaceutically acceptable salt thereof.

In a fifth preferred embodiment the benzoimidazole derivative of the invention is a compound of Formula III,

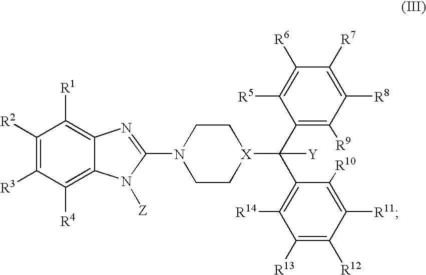

an isomer thereof or a mixture of its isomers, or a pharmaceutically acceptable salt thereof, wherein

R¹, R², R³, R⁴, R⁵, R⁶, R⁷, R⁸, R⁹, R¹⁰, R¹¹, R¹², R¹³ and R¹⁴ independently of each other, represent hydrogen, halo, trifluoromethyl, trifluoromethoxy, cyano, alkyl, alkoxy, amino, N-alkyl-amino or N,N-dialkyl-amino;

Z represents hydrogen, alkyl or benzyl, which benzyl may optionally be substituted one or more times with halo, trifluoromethyl, trifluoromethoxy, cyano, alkyl, alkoxy, amino, N-alkyl-amino and/or N,N-dialkyl-amino;

X represents CH or N; and

Y represents hydrogen, fluoro, hydroxy or alkoxy.

In a more preferred embodiment R¹, R², R³, R⁴, R⁵, R⁶, R⁷, R⁸, R⁹, R¹⁰, R¹¹, R¹², R¹³ and R¹⁴, independently of each other, represent hydrogen, halo, trifluoromethyl, trifluoromethoxy, cyano, alkyl, alkoxy, amino, N-alkyl-amino or N,N-dialkyl-amino.

In a more preferred embodiment R¹, R², R³, R⁴, R⁵, R⁶, R⁷, R⁸, R⁹, R¹⁰, R¹¹, R¹², R¹³ and R¹⁴, independently of each other, represent hydrogen, halo or trifluoromethyl.

In a still more preferred embodiment R¹, R², R³, R⁴, R⁵, R⁶, R⁷, R⁸, R⁹, R¹⁰, R¹¹, R¹², R¹³ and R¹⁴, independently of each other, represent hydrogen or halo, and in particular fluoro or chloro.

In another more preferred embodiment Z represents hydrogen, alkyl or benzyl, which benzyl may optionally be substituted one or more times with halo, trifluoromethyl, trifluoromethoxy, cyano, alkyl, alkoxy, amino, N-alkyl-amino and/or N,N-dialkyl-amino.

In a more preferred embodiment Z represents hydrogen or alkyl.

In an even more preferred embodiment Z represents hydrogen.

In a third more preferred embodiment X represents CH or N. In an even more preferred embodiment X represents CH. In a still more preferred embodiment X represents N.

In a fourth more preferred embodiment Y represents hydrogen, fluoro, hydroxy or alkoxy. In a more preferred embodiment Y represents hydrogen or hydroxy.

In a most preferred embodiment the benzoimidazole derivative of the invention is
[1-(1H-Benzoimidazol-2-yl)-piperidin-4-yl]diphenyl methanol;
2-(4-Benzhydryl-piperidin-1-yl)-1H-benzoimidazole;
2-(4-Benzhydrylpiperazin-1-yl)-1H-benzoimidazole; or
2-{4-[Bis-(4-fluorophenyl)methyl]piperazin-1-yl}-1H-benzoimidazole;

or an enantiomers or a mixture of its enantiomers, or a pharmaceutically acceptable salt thereof.

In a sixth preferred embodiment the benzoimidazole derivative of the invention is a compound of Formula IV,

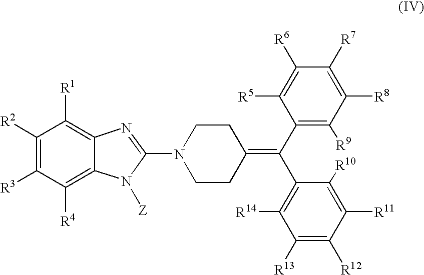

an isomer thereof or a mixture of its isomers, or a pharmaceutically acceptable salt thereof, wherein

R¹, R², R³, R⁴, R⁵, R⁶, R⁷, R⁸, R⁹, R¹⁰, R¹¹, R¹², R¹³ and R¹⁴, independently of each other, represent hydrogen, halo, trifluoromethyl, trifluoromethoxy, cyano, alkyl, alkoxy, amino, N-alkyl-amino or N,N-dialkyl-amino; and

Z represents hydrogen, alkyl or benzyl, which benzyl may optionally be substituted one or more times with halo, trifluoromethyl, trifluoromethoxy, cyano, alkyl, alkoxy, amino, N-alkyl-amino and/or N,N-dialkyl-amino.

In a more preferred embodiment R¹, R², R³, R⁴, R⁵, R⁶, R⁷, R⁸, R⁹, R¹⁰, R¹¹, R¹², R¹³ and R¹⁴, independently of each other, represent hydrogen, halo, trifluoromethyl, trifluoromethoxy, cyano, alkyl, alkoxy, amino, N-alkyl-amino or N,N-dialkyl-amino.

In a more preferred embodiment R¹, R², R³, R⁴, R⁵, R⁶, R⁷, R⁸, R⁹, R¹⁰, R¹¹R¹², R¹³ and R¹⁴, independently of each other, represent hydrogen, halo or trifluoromethyl.

In a still more preferred embodiment R¹, R², R³, R⁴, R⁵, R⁶, R⁷, R⁸, R⁹, R¹⁰, R¹¹, R¹² and R¹³, independently of each other, represent hydrogen or halo, and in particular fluoro or chloro.

In another more preferred embodiment Z represents hydrogen, alkyl or benzyl, which benzyl may optionally be substituted one or more times with halo, trifluoromethyl, trifluoromethoxy, cyano, alkyl, alkoxy, amino, N-alkyl-amino and/or N,N-dialkyl-amino.

In a more preferred embodiment Z represents hydrogen or alkyl. In an even more preferred embodiment Z represents hydrogen.

In a most preferred embodiment the benzoimidazole derivative of the invention is
2-(4-Benzhydrylidene-piperidin-1-yl)-1H-benzoimidazole;

or an enantiomers or a mixture of its enantiomers, or a pharmaceutically acceptable salt thereof.

Any combination of two or more of the embodiments described herein is considered within the scope of the present invention.

### Definition Of Substituents

In the context of this invention halo represents fluoro, chloro, bromo or iodo. Thus a trihalomethyl group represents e.g. a trifluoromethyl group, a trichloromethyl group, and similar trihalo-substituted methyl groups.

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention an alkoxy group designates an “alkyl-O—” group, wherein alkyl is as defined above. Examples of preferred alkoxy groups of the invention include methoxy and ethoxy.

In the context of this invention an N-alkyl-amino group designates a (secondary) amino group, monosubstituted with an alkyl group as defined above.

In the context of this invention an N,N-dialkyl-amino group designates a (tertiary) amino group, disubstituted with alkyl groups as defined above.

### Pharmaceutically Acceptable Salts

The chemical compound of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a chemical compound of the invention include, without limitation, the sodium, the potassium, the calcium, the magnesium, the zinc, the aluminium, the lithium, the choline, the lysine, and the ammonium salt, and the like, of a chemical compound of the invention containing an anionic group. Such cationic salts may be formed by procedures well known and described in the art.

### Isomers

It will be appreciated by those skilled in the art that the compounds of the present invention may exist in different stereoisomeric forms, including enantiomers, diastereomers, as well as geometric isomers (cis-trans isomers). The invention includes all such isomers and any mixtures thereof including racemic mixtures.

Racemic forms can be resolved into the optical antipodes by known methods and techniques. One way of separating the enantiomeric compounds (including enantiomeric intermediates) is by use of an optically active amine, and liberating the diastereomeric, resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L-(tartrates, mandelates, or camphorsulphonate) salts for example.

Additional methods for the resolving the optical isomers are known in the art. Such methods include those described by Jaques J. Collet A, & Wilen S in “Enantiomers, Racemates, and Resolutions”, John Wiley and Sons, New York (1981).

Optical active compounds can also be prepared from optical active starting materials or intermediates.

### Prodrugs

The benzoimidazole derivative of the invention may optionally be administered in the form of a suitable prodrug. In the context of this invention the term “prodrug” denotes a compound, which is a drug precursor and which, following administration and absorption, release the drug in vivo via some metabolic process.

Particularly favoured prodrugs are those that increase the bioavailability of the compounds of the invention, e.g. by allowing an orally administered compound to be more readily absorbed into the blood, or which enhance delivery of the parent compound to a specific biological compartment, e.g. the brain or lymphatic system.

Thus examples of suitable prodrugs of the benzoimidazole derivative of the invention include compounds modified at one or more reactive or derivatizable groups of the parent compound. Of particular interest are compounds modified at a carboxyl group, a hydroxyl group, or an amino group. Examples of suitable derivatives are esters or amides.

### Methods of Preparation

The benzoimidazole derivatives of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

Three subtypes of small-conductance calcium-activated potassium channels (SK channels) have been identified, i.e. SK1, SK2 and SK3 (corresponding to KCNN1-3 using the genomic nomenclature). The novel benzoimidazole derivatives of the invention are found to be potent inhibitors of the SK channels, including SK1, SK2, and in SK3.

Moreover, preferred compounds of the invention show a dual activity of being a potent monoamine reuptake inhibitor and an inhibitor of small-conductance calcium-activated potassium channels (SK channels). Preferred compounds of the invention show a dual biological activity at the sub-micromolar level (i.e. below 1 μM), preferably at the low nanomolar level (i.e. below 0.1 μM).

Due to their biological activity the benzoimidazole derivatives of the invention may be used for the treatment, prevention or alleviation of a disease or a disorder or a condition of a mammal, including a human, which disease, disorder or condition is responsive to inhibition of monoamine neurotransmitter re-uptake in the central nervous system and/or inhibition of SK_{Ca} channels.

Such diseases, disorders and conditions include depression, pseudodementia, Ganser's syndrome, obsessive compulsive disorder (OCD), panic disorder, memory deficits, memory loss, attention deficit hyperactivity disorder, obesity, anxiety, eating disorder, Parkinson's disease, parkinsonism, dementia, dementia of ageing, senile dementia, acquired immunodeficiency syndrome dementia complex, memory dysfunction in ageing, social phobia, drug addiction, drug misuse, cocaine abuse, tobacco abuse, alcoholism, pain, migraine pain, bulimia, premenstrual syndrome, late luteal phase syndrome, post-traumatic syndrome, chronic fatigue syndrome, premature ejaculation, erectile difficulty, anorexia nervosa, sleep disorders, autism, mutism, trichotillomania, narcolepsy, Gilles de la Tourettes disease, inflammatory bowel disease or irritable bowel syndrome.

In a preferred embodiment the disease, disorder or condition is depression, obsessive-compulsive disorder (OCD), mood disorders, body dysmorphic disorder, bulimia nervosa, premenstrual dysphoric disorder, panic disorder, ADHD, eating disorders, anxiety, anxiety disorders, panic disorders, panic attacks, phobias, irritable bowel syndrome (IBS) or premature ejaculation.

In another preferred embodiment the disease, disorder or condition is depression, pseudodementia, Ganser's syndrome, obsessive compulsive disorders (OCD), panic disorders, memory deficits, attention deficit hyperactivity disorder, obesity, anxiety, an eating disorder or Parkinson's disease.

### Methods of Therapy

In another aspect the invention provides a method for the treatment or alleviation of diseases or disorders or conditions of living animal bodies, including humans, which disease, disorder or condition is responsive to inhibition of monoamine neurotransmitter re-uptake in the central nervous system and/or inhibition of SK_{Ca} channels.

Preferred medical indications contemplated according to the invention are those stated above.

### BRIEF DESCRIPTION OF THE DRAWINGS

The present invention is further illustrated by reference to the accompanying drawing, in which:

FIG. 1 shows the 5HT syndrome in NMRI mice (n=4); 5-HT syndrome score (Max=16) vs. Time (min.), after s.c. administration of 50 mg/ml nialamid at t=−120 min, followed by p.o. administration of the test compound (0.3, 1, 3 mg/kg) and/or citalopram (1 mg/kg); and

FIG. 2 shows the PBZ ptosis in NMRI mice (n=6); Score (ptosis max=24; Bison max=6) vs. dose of the test compound (0.3, 1 and 3 mg/kg) at t=−60 min, and p.o. administration of reboxetine (0.1 mg/kg) at t=−60 min, followed by i.p. administration of 40 mg/kg TBZ at t=−30 min.

### EXAMPLES

The invention is further illustrated with reference to the following examples, which are not intended to be in any way limiting to the scope of the invention as claimed.

General: The procedures represent generic procedures used to prepare compounds of the invention. Abbreviations used are as follows:
Ac: acetyl

### DMF: N,N-dimethylformamide

DMSO: dimethylsulfoxide
Et: ethyl
eq: equivalent(s)
LCMS: liquid chromatography mass spectrometry
Me: methyl
mp: melting point
MW: microwave
rt: room temperature

### Procedure A

2-Chlorobenzimidazole and the required piperidine or piperazine derivative (commercially available or prepared via literature procedure) were suspended in acetonitrile in a closed vial and heated to 170-200° C. for 20-40 min by use of MW irradiation. After cooling to rt the precipitated solid was filtered off, washed with acetonitrile and recrystallised to give the desired product. Alternatively, the crude product from the reaction mixture was purified by column chromatography or by preparative LCMS to give the desired product as the free base.

An example of Procedure A, the preparation of 2-[4-(3,4-dichlorobenzyl)piperidin-1-yl]-1H-benzoimidazole, is shown in Scheme 1.

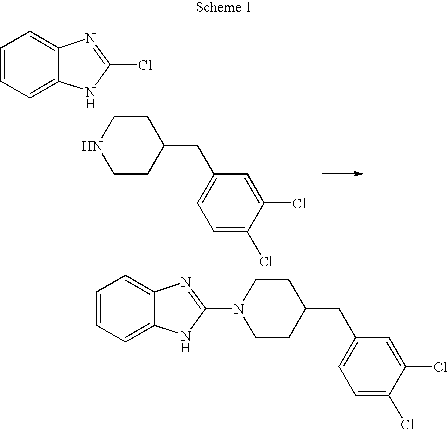

### Procedure B

A stirred solution of 2-chlorobenzimidazole in dry DMF was (under N₂ atmosphere) cooled to 0° C. and NaH (1.3 eq) was added. After stirring for 30 min at rt the required benzyl halide was added dropwise and the reaction mixture stirred at rt overnight. Saturated aqueous NaHCO₃ was added and the mixture extracted with EtOAc. The combined organic phases were dried (MgSO₄), filtered and concentrated in vacuo to give the desired 2-chloro-1-benzylbenzoimidazole derivative. This intermediate was subsequently dissolved in acetonitrile, added the required piperidine or piperazine derivative (1-2 eq) and heated by means of MW irradiation at 190-200° C. in a sealed vial for 15-40 min. The reaction mixture was evaporated to dryness and the crude product purified by preparative LCMS or column chromatography to give the desired product as the free base.

An example of Procedure B, the preparation of 2-(4-benzylpiperidin-1-yl)-1-(3,4-difluorobenzyl)-1H-benzoimidazole, is shown in Scheme 2.

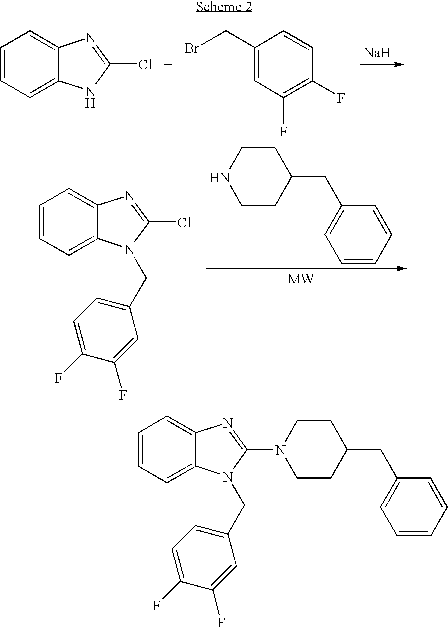

### Example 1

### 2-[4-(3,4-Dichlorobenzyl)piperidin-1-yl]-1H-benzoimidazole

The title compound was prepared as described in Procedure A. The precipitated solid from the reaction mixture was filtered off and washed with acetonitrile to give the title compound as a hydrogen chloride salt (mp 268-270° C.). MS (ES⁺) m/z 360 (M⁺, 100).

### Example 2

### 2-(4-Benzyl piperidin-1-yl)-1H-benzoimidazole

The title compound was prepared as described in Procedure A. The precipitated solid from the reaction mixture was filtered off and washed with acetonitrile to give the title compound as the free base (mp 193-194° C.). MS (ES⁺) m/z 292 ([M+1]⁺, 100).

### Example 3

### [1(1H-Benzoimidazol-2-yl)-piperidin-4-yl]diphenylmethanol

The title compound was prepared from 2-chlorobenzimidazole and commercially available α-(4-piperidyl)benzhydrol as described in Procedure A. The title compound was isolated upon basic aqueous work-up as the free base (mp 237-239° C.). MS (ES⁺) m/z 384 ([M+1]⁺, 100).

### Example 4

### 2-(4-Benzylpiperidin-1-yl)-1-[4-chloro-3-(trifluoromethyl)benzyl]-1H-benzoimidazole

The title compound was prepared in two steps as described in Procedure B. The crude product was purified by preparative LCMS to give the title compound as the free base (mp 124-125.5° C.). MS (ES⁺) m/z 484 (M⁺, 100).

### Example 5

### 2-(4-Benzylpiperidin-1-yl)-1-(3,4-difluorobenzyl)-1H-benzoimidazole

The title compound was prepared in two steps as described in Procedure B. The crude product was purified by preparative LCMS to give the title compound as the free base (mp 155-155.5° C.). MS (ES⁺) m/z 418 ([M+1]⁺, 100).

### Example 6

### 2-(4-Benzhydrylpiperazin-1-yl)-1H-benzoimidazole

The title compound was prepared from 2-chlorobenzimidazole and commercially available 1-(diphenylmethyl)piperazine as described in Procedure A. The precipitated solid from the reaction mixture was filtered off to give the title compound as the free base (mp>230° C. (decomp.)). MS (ES⁺) m/z 369 ([M+1]⁺, 100).

### Example 7

### 2-(4-Benzylpiperazin-1-yl)-1H-benzoimidazole

The title compound was prepared as described in Procedure A and isolated upon basic aqueous work-up as the free base (mp 235-237° C.). MS (ES⁺) m/z 369 ([M+1]⁺, 100).

### Example 8

### 1-(3,4-Difluorobenzyl)-2-[4-(3,4-difluorobenzyl)piperazin-1-yl]-1H-benzoimidazole

The title compound was prepared in two steps as described in Procedure B. The crude product was purified by preparative LCMS to give the title compound as the free base (yellowish gum). ¹NMR (CDCl₃) δ 2.56 (br s, 4H), 3.25-3.30 (m, 4H), 3.51 (s, 2H), 5.15 (s, 2H), 6.86-6.91 (m, 1H), 6.96-7.24 (m, 8H), 7.65 (d, 1H). MS (ES⁺) m/z 455 ([M+1]⁺, 100).

### Example 9

### 2-{4-[Bis-(4-fluorophenyl)methyl]piperazin-1-yl}-1H-benzoimidazole

The title compound was prepared from 2-chlorobenzimidazole and commercially available 1-(4,4′-difluorobenzhydryl)piperazine as described in Procedure A. The precipitated solid from the reaction mixture was filtered off to give the title compound as the hydrochloride salt (mp>240° C. (decomp.)). MS (ES⁺) m/z 405 ([M+1]⁺, 100).

### Example 10

### 2-[4-(3,4-Dichlorophenoxy)piperidin-1-yl]-1H-benzoimidazole

The title compound was prepared from 2-chlorobenzimidazole and 4-(3,4-dichlorophenoxy)piperidine as described in Procedure A. The precipitated solid from the reaction mixture was filtered off and washed with acetonitrile to give the title compound as the hydrochloride salt (mp 297-298° C.). MS (ES⁺) m/z 363 ([M+1]⁺, 100).

### Example 11

### 2-[4-(3,4-Dichlorophenylsulfanyl)piperidin-1-yl]-1H-benzoimidazole

The title compound was prepared from 2-chlorobenzimidazole and 4-(3,4-dichlorophenylsulfanyl)piperidine as described in Procedure A. The crude product was purified by preparative LCMS to give the title compound as the free base. ¹NMR (DMSO-d6) δ 1.50-1.62 (m, 2H), 1.95-2.03 (m, 2H), 3.12-3.21 (m, 2H), 3.65-3.70 (m, 1H), 3.95-4.04 (m, 2H), 6.85-6.96 (m, 2H), 7.10-7.21 (m, 2H), 7.41 (d, 1H), 7.58 (d, 1H), 7.69 (s, 1H), 11.3 (s, 1H). MS (ES⁺) m/z 378 (M⁺, 100).

### Example 12

### 2-(4-Benzhydrylidene-piperidin-1-yl)-1H-benzoimidazole

[1-(1H-Benzoimidazol-2-yl)-piperidin-4-yl]diphenylmethanol (prepared as described above) was dissolved in trifluoroacetic acid and stirred for 1 h at rt. The reaction mixture was evaporated to dryness, added saturated aqueous NaHCO₃ and extracted with EtOAc. The combined organic phases were dried (MgSO₄), filtered and concentrated in vacuo to give the crude product which was recrystallized from MeOH/water to give the title compound as the free base (mp 229-230° C.). MS (ES⁺) m/z 366 ([M+1]⁺, 100).

### Example 13

### 2-(4-Benzhydryl-piperidin-1-yl)-1H-benzoimidazole

2-(4-Benzhydrylidene-piperidin-1-yl)-1H-benzoimidazole (prepared as described above) was dissolved in ethanol, added a catalytic amount of 10% Pd/C and hydrogenated at rt with hydrogen gas. The reaction mixture was filtered through celite, evaporated to dryness and the crude product purified by LCMS to give the title compound as the free base (mp 256-258° C.). MS (ES⁺) m/z 368 ([M+1]⁺, 100).

### Example 14

### 2-[4-(3,4-Difluorobenzyl)piperidin-1-yl]-1H-benzoimidazole

The title compound was prepared as described in Procedure A. The precipitated solid from the reaction mixture was filtered off and washed with acetonitrile to give the title compound as a hydrogen chloride salt. MS (ES⁺) m/z 328 ([M+1]⁺, 100). HR-MS: 328.1621 ([M+1]⁺, C₁₉H₂₀F₂N₃; calc. 328.162528).

### Example 15

### Biological Activity

Preclinical data in our laboratories has shown that the combination of a selective inhibitor of SK channels, 1,3-Bis-(3,4-difluoro-benzyl)-1,3-dihydro-benzoimidazol-2-ylideneamine, hereafter designated the test compound, showing more than 100 fold selectivity for inhibition of SK3 channels in patchclamp electrophysiology over inhibiton of [3H]DA, [3H]5-HT and [3H]NA reuptake in vitro), with a sub-threshold dose of the selective serotonin reuptake inhibitor (SSRI), citalopram, enhanced the ability of the latter to induce symptoms analagous to the serotonin syndrome, following pre-treatment with the monoamine amine oxidase inhibitor, nialamide. The nialamide induced 5-HT syndrome paradigm is widely considered to reflect the ability of a compound to inhibit the reuptake of 5-HT from the synapse.

Method: Mice were administered nialamide (50 mg/kg, s.c., −120 min) followed by the test compound (0.3-3 mg/kg, i.p.) and citalopram (1 mg/kg, p.o) at time-point 0 min. The presence of head twitches, hindlimb abduction, head weaving, and piano playing behaviours were then scored by a trained observer using a recognised rating scale: maximum score/mouse=4, minimum score/mouse=0.

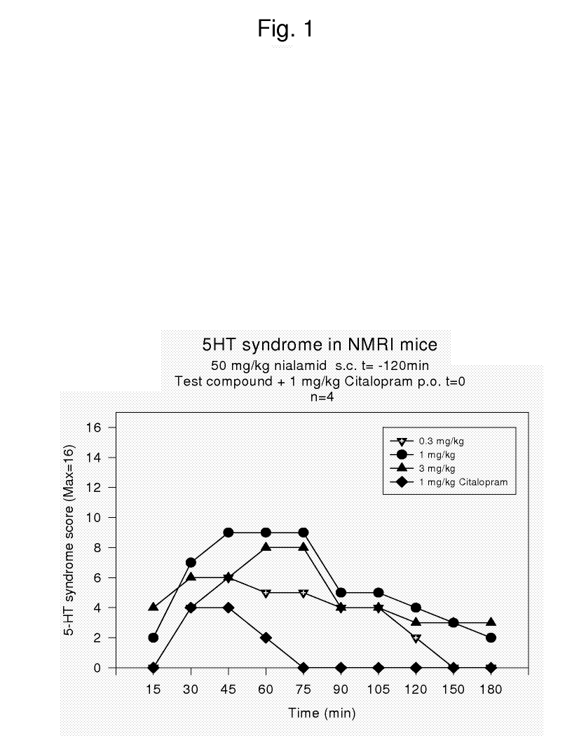

The results of this experiment are presented in FIG. 1.

Thus, this data suggested that blockade of SK channels in combination with the SSRI induced a superior monoamine neurotransmission to that of the SSRI alone.

In keeping with this theory, data from our laboratories has also shown that the combination of an inhibitor of SK channels with a sub-threshold dose of the noradrenaline reuptake inhibitor (NRI), reboxetine, enhanced the ability of the latter to reverse tetrabenazine-incuced ptosis. Reversal of tetrabenazine-induced ptosis is widely considered to reflect the ability of a compound to inhibit the reuptake of noradrenaline from the synapse.

Method: Mice were administered the test compound (0.3-3 mg/kg, i.p.) and reboxetine (0.1 mg/kg, p.o.) at timepoint −60 min, followed by tetrabenazine (40 mg/kg, i.p., −30 min). The presence of ptosis was then scored by a trained observer using a recognised rating scale: maximum score/mouse=4, minimum score/mouse=0.

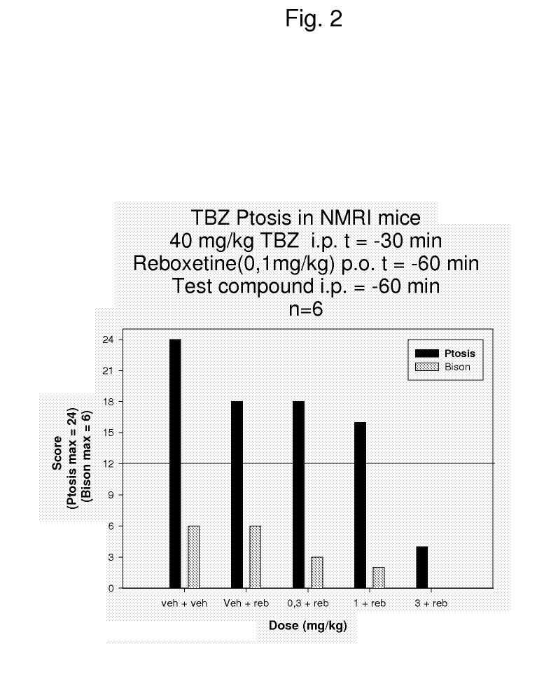

The results of this experiment are presented in FIG. 2.

Thus, this data suggested that blockade of SK channels in combination with the NRI induced a superior monoamine neurotransmission to that of the NRI alone.

1-25. (canceled)

26. A pharmaceutical composition comprising a therapeutically effective amount of an active pharmaceutical ingredient selected from
A) a monoamine reuptake inhibitor; and
B) an SK inhibitor;
together with one or more adjuvants, excipients, carriers and/or diluents.

27. The pharmaceutical composition of claim 26, comprising a compound having the dual activity of a monoamine reuptake inhibitor and an SK inhibitor as the only API.

28. The use of
A) a compound selected from the group of monoamine reuptake inhibitors; and
B) a compound selected from the group of SK inhibitors;
for the manufacture of a pharmaceutical composition/medicament.

29. A benzoimidazole derivative of Formula I 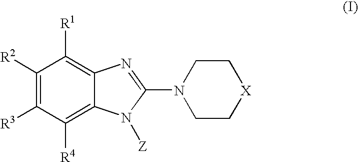
a stereoisomer thereof or a mixture of its stereoisomers, or a pharmaceutically acceptable salt thereof, wherein
R¹, R², R³ and R⁴, independently of each other, represent hydrogen, halo, trifluoromethyl, trifluoromethoxy, cyano, alkyl, alkoxy, amino, N-alkyl-amino or N,N-dialkyl-amino;
Z represents hydrogen, alkyl or benzyl, which benzyl may optionally be substituted one or more times with halo, trifluoromethyl, trifluoromethoxy, cyano, alkyl, alkoxy, amino, N-alkyl-amino and/or N,N-dialkyl-amino; and
X represents CH-A′, N-A′, or C=A″; wherein
A′ represents a group of Formula Ia or Ib: 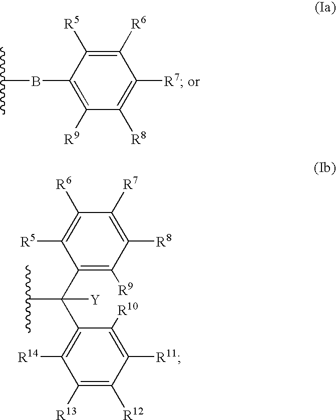
wherein
B represents CH₂, O or S;
Y represents hydrogen, fluoro, hydroxy or alkoxy; and
R⁵, R⁶, R⁷, R⁸, R⁹, R¹⁰, R¹¹, R¹², R¹³ and R¹⁴, independently of each other, represent hydrogen, halo, trifluoromethyl, trifluoromethoxy, cyano, alkyl, alkoxy, amino, N-alkyl-amino or N,N-dialkyl-amino; and
A″ represents a group of Formula Ic: 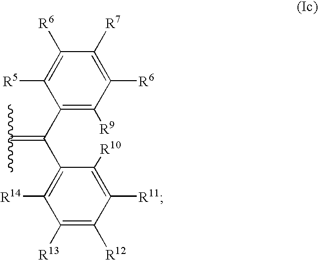
wherein
R⁵, R⁶, R⁷, R⁸, R⁹, R¹⁰, R¹¹, R¹², R¹³ and R¹⁴, independently of each other, represent hydrogen, halo, trifluoromethyl, trifluoromethoxy, Cyano, alkyl, alkoxy, amino, N-alkyl-amino or N,N-dialkyl-amino.

30. The benzoimidazole derivative of claim 29, a stereoisomer thereof or a mixture of its stereoisomers, or a pharmaceutically acceptable salt thereof, wherein
R¹, R², R³ and R⁴, independently of each other, represent hydrogen, halo, trifluoromethyl, trifluoromethoxy, cyano, alkyl, alkoxy, amino, N-alkyl-amino or N,N-dialkyl-amino.

31. The benzoimidazole derivative of claim 29, a stereoisomer thereof or a mixture of its stereoisomers, or a pharmaceutically acceptable salt thereof, wherein
Z represents hydrogen, alkyl or benzyl, which benzyl may optionally be substituted one or more times with halo, trifluoromethyl, trifluoromethoxy, cyano, alkyl, alkoxy, amino, N-alkyl-amino and/or N,N-dialkyl-amino.

32. The benzoimidazole derivative of claim 29, a stereoisomer thereof or a mixture of its stereoisomers, or a pharmaceutically acceptable salt thereof, wherein
X represents CH-A′ or N-A′, and
A′ is as defined in claim 29.

33. The benzoimidazole derivative of claim 29, represented by Formula II 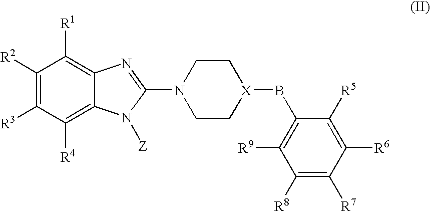
a stereoisomer thereof or a mixture of its stereoisomers, or a pharmaceutically acceptable salt thereof, wherein
R¹, R², R³, R⁴, R⁵, R⁶, R⁷, R⁸ and R⁹, independently of each other, represent hydrogen, halo, trifluoromethyl, trifluoromethoxy, cyano, alkyl, alkoxy, amino, N-alkyl-amino or N,N-dialkyl-amino;
Z represents hydrogen, alkyl or benzyl, which benzyl may optionally be substituted one or more times with halo, trifluoromethyl, trifluoromethoxy, cyano, alkyl, alkoxy, amino, N-alkyl-amino and/or N,N-dialkyl-amino;
X represents CH or N; and
B represents CH₂, O or S.

34. The benzoimidazole derivative of claim 33, a stereoisomer thereof or a mixture of its stereoisomers, or a pharmaceutically acceptable salt thereof, wherein
R¹, R², R³, R⁴, R⁵, R⁶, R⁷, R⁸ and R⁹, independently of each other, represent hydrogen, halo, trifluoromethyl, trifluoromethoxy, cyano, alkyl, alkoxy, amino, N-alkyl-amino or N,N-dialkyl-amino.

35. The benzoimidazole derivative of claim 33, a stereoisomer thereof or a mixture of its stereoisomers, or a pharmaceutically acceptable salt thereof, wherein
Z represents hydrogen, alkyl or benzyl, which benzyl may optionally be substituted one or more times with halo, trifluoromethyl, trifluoromethoxy, cyano, alkyl, alkoxy, amino, N-alkyl-amino and/or N,N-dialkyl-amino.

36. The benzoimidazole derivative of claim 33, a stereoisomer thereof or a mixture of its stereoisomers, or a pharmaceutically acceptable salt thereof, wherein
X represents CH or N.

37. The benzoimidazole derivative of claim 33, a stereoisomer thereof or a mixture of its stereoisomers, or a pharmaceutically acceptable salt thereof, wherein
B represents CH₂, O or S.

38. The benzoimidazole derivative of claim 29, represented by Formula III 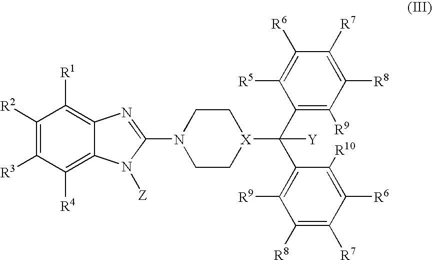
a stereoisomer thereof or a mixture of its stereoisomers, or a pharmaceutically acceptable salt thereof, wherein
R¹, R², R³, R⁴, R⁵, R⁶, R⁷, R⁸, R⁹, R¹⁰, R¹¹, R¹², R¹³ and R¹⁴, independently of each other, represent hydrogen, halo, trifluoromethyl, trifluoromethoxy, cyano, alkyl, alkoxy, amino, N-alkyl-amino or N,N-dialkyl-amino;
Z represents hydrogen, alkyl or benzyl, which benzyl may optionally be substituted one or more times with halo, trifluoromethyl, trifluoromethoxy, cyano, alkyl, alkoxy, amino, N-alkyl-amino and/or N,N-dialkyl-amino;
X represents CH or N; and
Y represents hydrogen, fluoro, hydroxy or alkoxy.

39. The benzoimidazole derivative of claim 38, a stereoisomer thereof or a mixture of its stereoisomers, or a pharmaceutically acceptable salt thereof, wherein
R¹, R², R³, R⁴, R⁵, R⁶, R⁷, R⁸, R⁹, R¹⁰, R¹¹, R¹², R¹³ and R¹⁴ independently of each other, represent hydrogen, halo, trifluoromethyl, trifluoromethoxy, cyano, alkyl, alkoxy, amino, N-alkyl-amino or N,N-dialkyl-amino.

40. The benzoimidazole derivative of claim 38, a stereoisomer thereof or a mixture of its stereoisomers, or a pharmaceutically acceptable salt thereof, wherein
Z represents hydrogen, alkyl or benzyl, which benzyl may optionally be substituted one or more times with halo, trifluoromethyl, trifluoromethoxy, cyano, alkyl, alkoxy, amino, N-alkyl-amino and/or N,N-dialkyl-amino.

41. The benzoimidazole derivative of claim 38, a stereoisomer thereof or a mixture of its stereoisomers, or a pharmaceutically acceptable salt thereof, wherein
X represents CH or N.

42. The benzoimidazole derivative of claim 38, a stereoisomer thereof or a mixture of its stereoisomers, or a pharmaceutically acceptable salt thereof, wherein
Y represents hydrogen, fluoro, hydroxy or alkoxy.

43. The benzoimidazole derivative of claim 29, represented by Formula IV 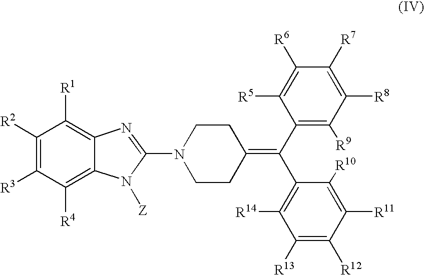
a stereoisomer thereof or a mixture of its stereoisomers, or a pharmaceutically acceptable salt thereof, wherein
R¹, R², R³, R⁴, R⁵, R⁶, R⁷, R⁸, R⁹, R¹⁰, R¹¹, R¹², R¹³ and R¹⁴ independently of each other, represent hydrogen, halo, trifluoromethyl, trifluoromethoxy, cyano, alkyl, alkoxy, amino, N-alkyl-amino or N,N-dialkyl-amino; and
Z represents hydrogen, alkyl or benzyl, which benzyl may optionally be substituted one or more times with halo, trifluoromethyl, trifluoromethoxy, cyano, alkyl, alkoxy, amino, N-alkyl-amino and/or N,N-dialkyl-amino.

44. The benzoimidazole derivative of claim 43, a stereoisomer thereof or a mixture of its stereoisomers, or a pharmaceutically acceptable salt thereof, wherein
R¹, R², R³, R⁴, R⁵, R⁶, R⁷, R⁸, R⁹, R¹⁰, R¹¹, R¹², R¹³ and R¹⁴ independently of each other, represent hydrogen, halo, trifluoromethyl, trifluoromethoxy, cyano, alkyl, alkoxy, amino, N-alkyl-amino or N,N-dialkyl-amino.

45. The benzoimidazole derivative of claim 43, a stereoisomer thereof or a mixture of its stereoisomers, or a pharmaceutically acceptable salt thereof, wherein
Z represents hydrogen, alkyl or benzyl, which benzyl may optionally be substituted one or more times with halo, trifluoromethyl, trifluoromethoxy, cyano, alkyl, alkoxy, amino, N-alkyl-amino and/or N,N-dialkyl-amino.

46. The benzoimidazole derivative of claim 29, which is
2-(4-Benzylpiperidin-1-yl)-1H-benzoimidazole;
2-(4-Benzylpiperidin-1-yl)-1-[4-chloro-3-(trifluoromethyl)benzyl]-1H-benzoimidazole;
2-(4-Benzylpiperidin-1-yl)-1-(3,4-difluorobenzyl)-1H-benzoimidazole;
2-[4-(3,4-Dichlorophenoxy)piperidin-1-yl]-1H-benzoimidazole;
2-[4-(3,4-Dichlorophenylsulfanyl)piperidin-1-yl]-1H-benzoimidazole;
2-[4-(3,4-Dichlorobenzyl)piperidin-1-yl]-1H-benzoimidazole;
2-(4-Benzylpiperazin-1-yl)-1H-benzoimidazole;
1-(3,4-Difluorobenzyl)-2-[4-(3,4-difluorobenzyl)piperazin-1-yl]-1H-benzoimidazole;
[1-(1H-Benzoimidazol-2-yl)-piperidin-4-yl]diphenylmethanol;
2-(4-Benzhydryl-piperidin-1-yl)-1H-benzoimidazole;
2-(4-Benzhydrylpiperazin-1-yl)-1H-benzoimidazole;
2-{4-[Bis-(4-fluorophenyl)methyl]piperazin-1-yl}-1H-benzoimidazole; or
2-(4-Benzhydrylidene-piperidin-1-yl)-1H-benzoimidazole;
or an enantiomers or a mixture of its enantiomers, or a pharmaceutically acceptable salt thereof.

47. A pharmaceutical composition comprising a therapeutically effective amount of a benzoimidazole derivative according to claim 29, or a pharmaceutically-acceptable salt thereof, together with one or more adjuvants, excipients, carriers and/or diluents.

48. The method according to claim 49, wherein the disease, disorder or conditions is depression, pseudodementia, Ganser's syndrome, obsessive compulsive disorder (OCD), panic disorder, memory deficits, memory loss, attention deficit hyperactivity disorder, obesity, anxiety, eating disorder, Parkinson's disease, parkinsonism, dementia, dementia of ageing, senile dementia, acquired immunodeficiency syndrome dementia complex, memory dysfunction in ageing, social phobia, drug addiction, drug misuse, cocaine abuse, tobacco abuse, alcoholism, pain, migraine pain, bulimia, premenstrual syndrome, late luteal phase syndrome, post-traumatic syndrome, chronic fatigue syndrome, premature ejaculation, erectile difficulty, anorexia nervosa, sleep disorders, autism, mutism, trichotillomania, narcolepsy, Gilles de la Tourettes disease, inflammatory bowel disease or irritable bowel syndrome.

49. A method of treatment, prevention or alleviation of a disease or a disorder or a condition of a living animal body, including a human, which disorder, disease or condition is responsive to inhibition of monoamine neurotransmitter re-uptake in the central nervous system and/or inhibition of SK_{Ca} channels, which method comprises the step of administering to such a living animal body in need thereof, a therapeutically effective amount of a benzoimidazole derivative according to claim 29, or a pharmaceutically-acceptable salt thereof.
